(19) United States
(12) Patent Application Publication
(10) Pub. No.: US 2016/0081540 A1
(43) Pub. Date: Mar. 24, 2016

(54) ENDOSCOPE CLEANING/DISINFECTING APPARATUS

(71) Applicant: OLYMPUS CORPORATION, Tokyo (JP)

(72) Inventor: Shintaro SUZUKI, Sagamihara-shi (JP)

(73) Assignee: OLYMPUS CORPORATION, Tokyo (JP)

(21) Appl. No.: 14/956,544

(22) Filed: Dec. 2, 2015

Related U.S. Application Data
(63) Continuation of application No. PCT/JP2014/075221, filed on Sep. 24, 2014.

(30) Foreign Application Priority Data
Jan. 24, 2014 (JP) 2014-011747

(51) Int. Cl. A61B 1/12; A61L 2/24; A61L 2/18; A61B 1/00

(52) U.S. Cl. CPC A61B 1/123; A61B 1/00059; A61L 2/24; A61L 2/18; A61B 2019/448

(57) ABSTRACT

An endoscope cleaning/disinfecting apparatus includes: a cleaning/disinfecting tank, a first detection section, a second detection section, a cleaning/disinfecting section, a cleaning/disinfecting detection section, an alerting section, a determination section that if, after reception of the endoscope in the cleaning/disinfecting tank is detected by the first detection section, removal of the endoscope from the cleaning/disinfecting tank is detected by the second detection section despite no detection of driving of the cleaning/disinfecting section by the cleaning/disinfecting detection section, determines that the uncleaned/undisinfected endoscope is removed from the cleaning/disinfecting tank, and a control section that if it is determined by the determination section that the uncleaned/undisinfected endoscope is removed, drives the alerting section to issue a warning.

### CROSS REFERENCE TO RELATED APPLICATION

This application is a continuation application of PCT/JP2014/075221 filed on Sep. 24, 2014 and claims benefit of Japanese Application No. 2014-011747 filed in Japan on Jan. 24, 2014, the entire contents of which are incorporated herein by this reference.

### BACKGROUND OF THE INVENTION

1. Field of the Invention

The present invention relates to an endoscope cleaning/disinfecting apparatus that cleans/disinfects an endoscope.

2. Description of the Related Art

In endoscope cleaning/disinfecting using an endoscope cleaning/disinfecting apparatus, an operator first puts a used endoscope in a cleaning/disinfecting tank and connects various tubes to fittings of the endoscope and then closes a cover and presses a start button. Then, a cleaning/disinfecting process starts, and after a lapse of a fixed time period, the cleaning/disinfecting process ends. Lastly, the operator opens the cover and removes the cleaned/disinfected endoscope.

Here, in the aforementioned cleaning/disinfecting process, after the used endoscope is put in the cleaning/disinfecting tank and the cover is closed or the cover is left open, in some cases, the endoscope is left for a long period of time without the start button being pressed.

In such cases, the uncleaned/undisinfected endoscope may be taken out by an operator that is different from an operator that put the endoscope in the cleaning/disinfecting tank, and thus, there is a demand for providing a function that informs the operator taking the endoscope out of the cleaning/disinfecting tank of the endoscope being uncleaned/undisinfected.

In view of such circumstances, the endoscope cleaning/disinfecting apparatus in Japanese Patent Application Laid-Open Publication No. 2003-235793 has a function that after an end of a cleaning/disinfecting process, transmits information that the relevant endoscope is a cleaned/disinfected one.

Furthermore, Japanese Patent Application Laid-Open Publication No. 2003-235793 discloses that a processor in an operating room has a function that when an endoscope is connected to the processor to use the endoscope, allows a microcomputer in the processor to receive information regarding whether or not the endoscope is a cleaned/disinfected one from the endoscope and if an uncleaned/undisinfected endoscope is connected to the processor, issues a warning. Consequently, the operator is informed of the endoscope being an uncleaned/undisinfected one.

### SUMMARY OF THE INVENTION

An endoscope cleaning/disinfecting apparatus according to an aspect of the present invention includes: a cleaning/disinfecting tank that is a container receiving an endoscope; a first detection section that detects reception of the endoscope by the cleaning/disinfecting tank; a second detection section that detects removal of the endoscope from the cleaning/disinfecting tank; a cleaning/disinfecting section for cleaning/disinfecting the endoscope inside the cleaning/disinfecting tank; a cleaning/disinfecting detection section that detects driving of the cleaning/disinfecting section; an alerting section that issues a warning; a determination section that is connected to the determination section is connected to the first detection section, the second detection section and the cleaning/disinfecting detection section, and if, after reception of the endoscope in the cleaning/disinfecting tank is detected by the first detection section, removal of the endoscope from the cleaning/disinfecting tank is detected by the second detection section despite no detection of driving of the cleaning/disinfecting section by the cleaning/disinfecting detection section, determines that the uncleaned/undisinfected endoscope is removed from the cleaning/disinfecting tank; and a control section that is connected to at least the determination section and the alerting section, and if it is determined by the determination section that the uncleaned/undisinfected endoscope is removed, drives the alerting section to issue a warning.

### BRIEF DESCRIPTION OF THE DRAWINGS

FIG. 1 is a diagram schematically illustrating an endoscope cleaning/disinfecting apparatus according to a first embodiment;

FIG. 2 is a block diagram schematically illustrating a warning section inside an apparatus body of the endoscope cleaning/disinfecting apparatus in FIG. 1;

FIG. 3 is a diagram illustrating an example of the cleaning/disinfecting section in the warning section in FIG. 2, together with an internal configuration of the cleaning/disinfecting apparatus;

FIG. 4 is a perspective view illustrating an endoscope guide member to be mounted in a cleaning/disinfecting tank in the endoscope cleaning/disinfecting apparatus in FIG. 1;

FIG. 5 is a perspective view illustrating the endoscope guide member in FIG. 4 mounted in the cleaning/disinfecting tank in the endoscope cleaning/disinfecting apparatus in FIG. 1;

FIG. 6 is a partial perspective view of the endoscope cleaning/disinfecting apparatus and an endoscope with the part enclosed by line X in FIG. 5 enlarged;

FIG. 7 is a block diagram of a warning section according to a modification in which the first detection section and the second detection section in FIG. 2 are provided separately;

FIG. 8 is a flowchart schematically illustrating a cleaning/disinfecting process of an endoscope using the endoscope cleaning/disinfecting apparatus in FIG. 1;

FIG. 9 is a flowchart indicating control operation performed by the control section in FIG. 2 to control a warning section;

FIG. 10 is a flowchart indicating a modification of control operation performed by the control section in FIG. 2 to control the warning section when an endoscope is detected again within a fixed time period after the warning step in FIG. 9;

FIG. 11 is a flowchart indicating a modification of the control operation performed by the control section in FIG. 2 to control the warning section when an endoscope is detected again within a fixed time period after the endoscope removal detection step in FIG. 9; and

FIG. 12 is a block diagram schematically illustrating a warning section inside an apparatus body of an endoscope cleaning/disinfecting apparatus according to a second embodiment.

### DETAILED DESCRIPTION OF THE PREFERRED EMBODIMENTS

Embodiments of the present invention will be described below with reference to the drawings. It should be noted that the drawings are schematic ones and, e.g., a relationship between a thickness and a width of each member and ratios in thickness among the respective members are different from actual ones, and it should be understood that parts that are different in dimensional relationship and ratio depending on the drawings are included in the drawings.

### First Embodiment

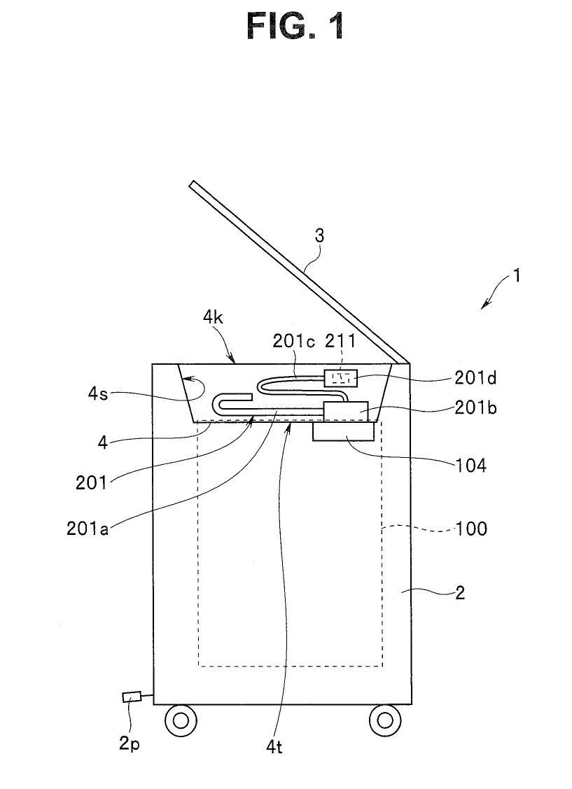

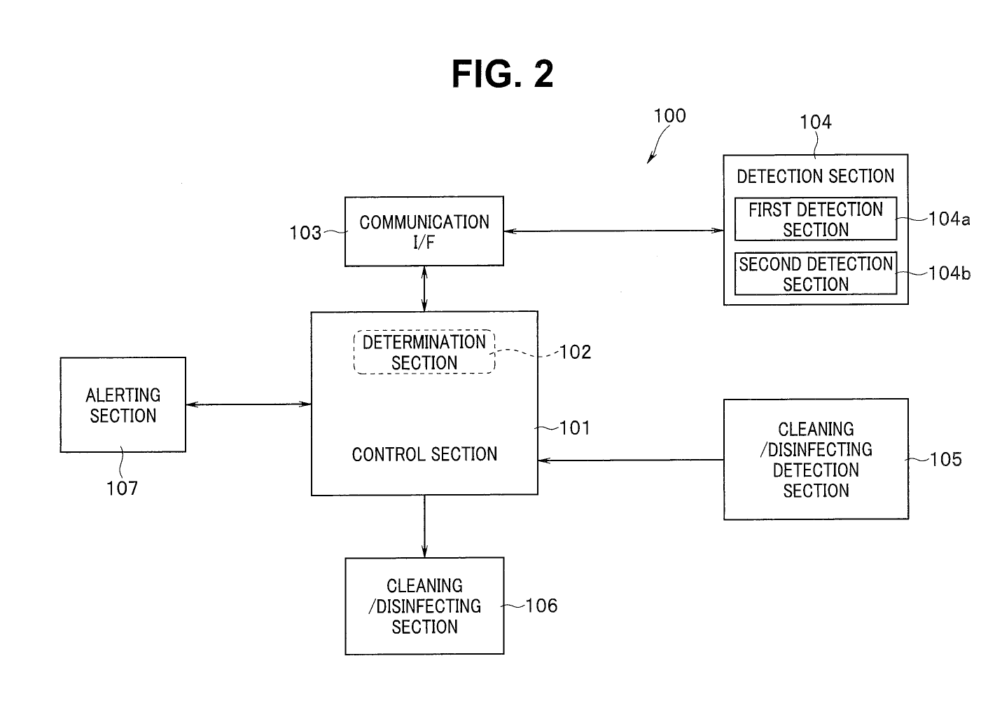

FIG. 1 is a diagram schematically illustrating an endoscope cleaning/disinfecting apparatus according to the present embodiment, and FIG. 2 is a block diagram schematically illustrating a warning section inside an apparatus body of the endoscope cleaning/disinfecting apparatus in FIG. 1.

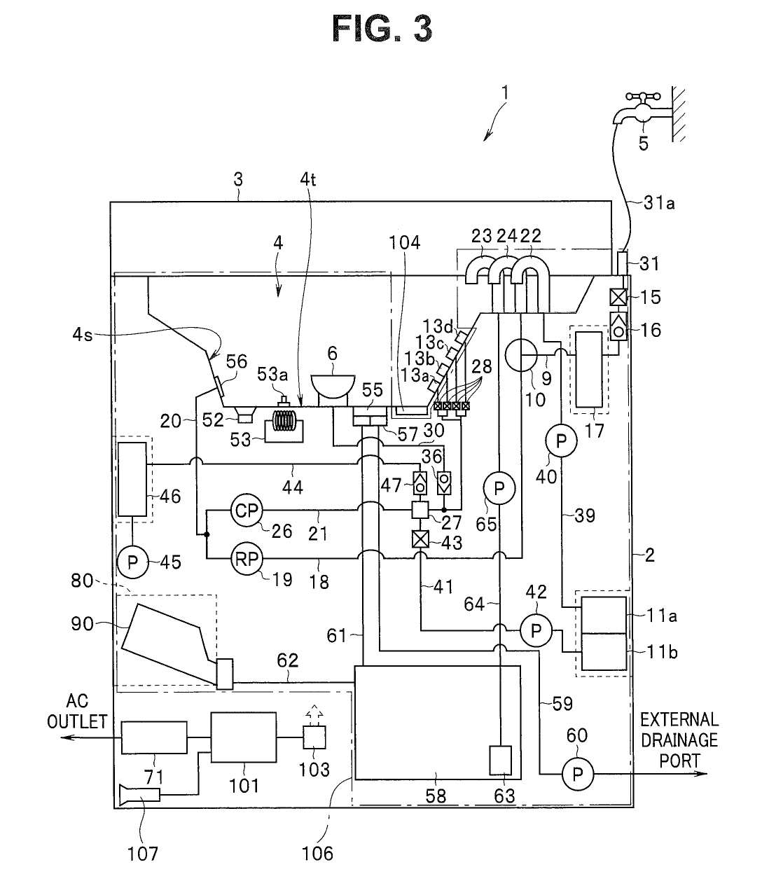

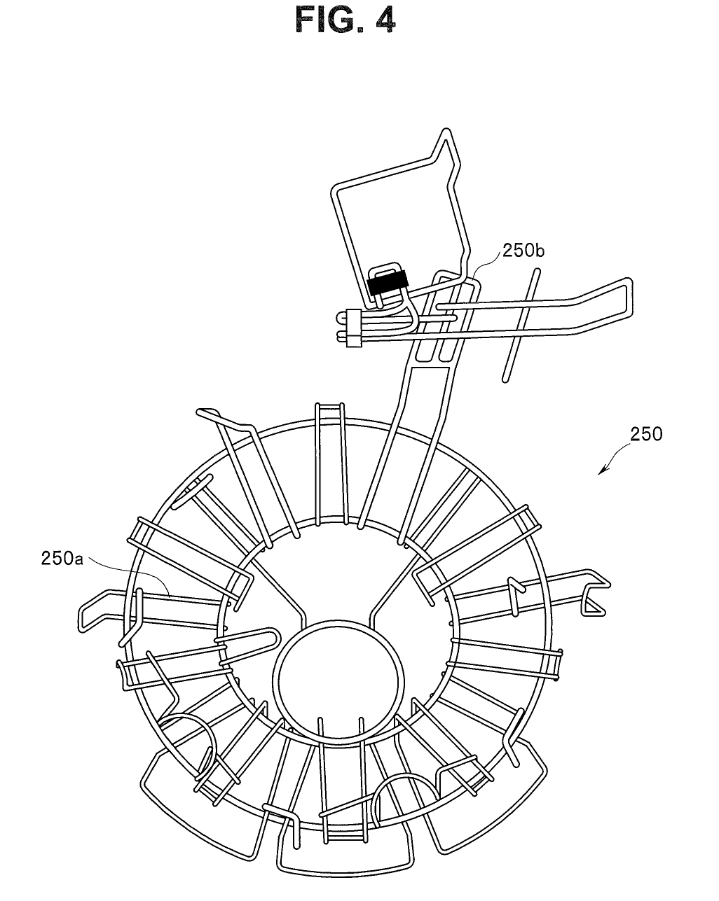

FIG. 3 is a diagram illustrating an example of the cleaning/disinfecting section in the warning section in FIG. 2, together with an internal configuration of the cleaning/disinfecting apparatus, and FIG. 4 is a perspective view illustrating an endoscope guide member to be mounted in a cleaning/disinfecting tank in the endoscope cleaning/disinfecting apparatus in FIG. 1.

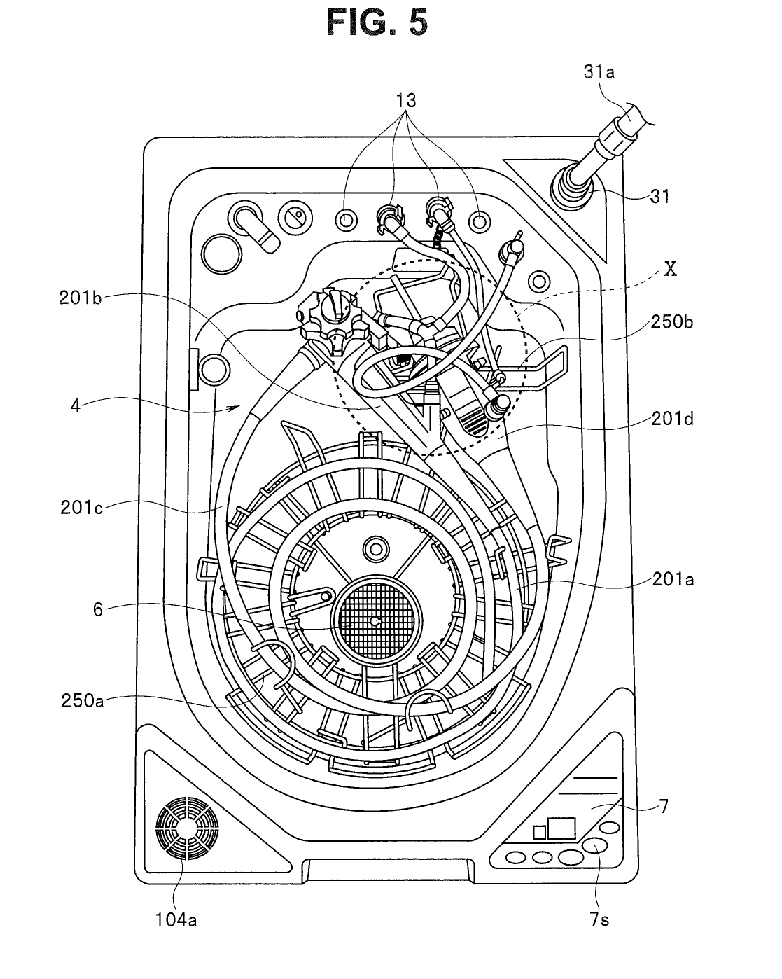

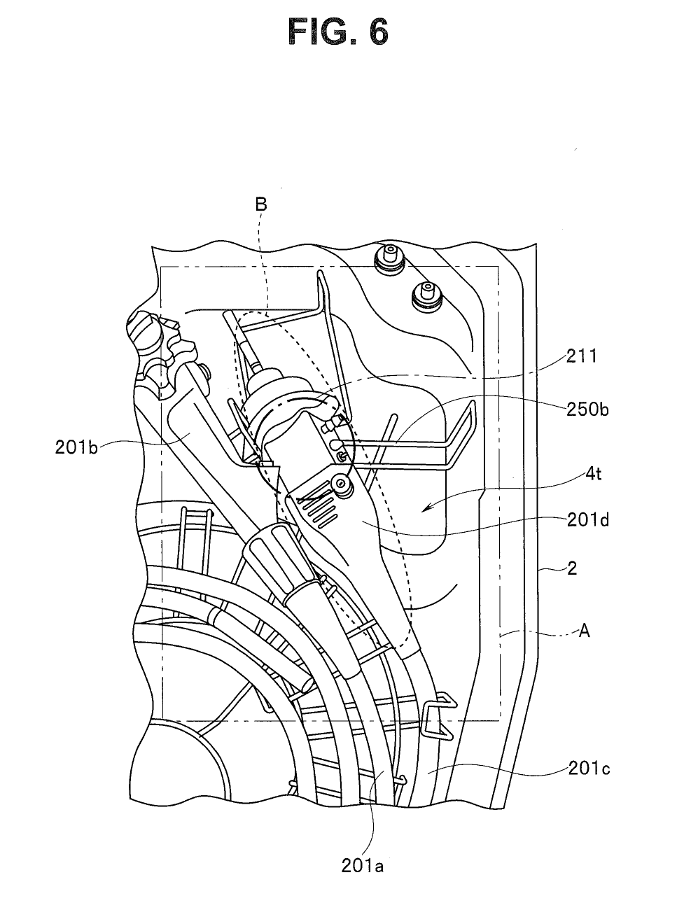

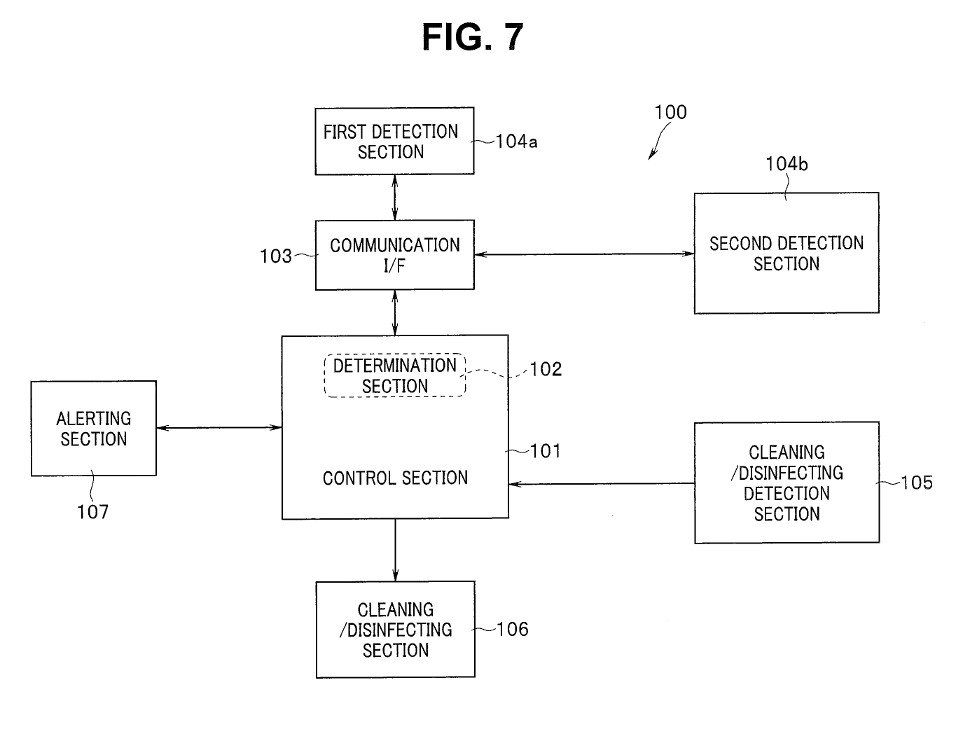

FIG. 5 is a perspective view illustrating the endoscope guide member in FIG. 4 mounted in the cleaning/disinfecting tank in the endoscope cleaning/disinfecting apparatus in FIG. 1, FIG. 6 is a partial perspective view of the endoscope cleaning/disinfecting apparatus and an endoscope with the part enclosed by line X in FIG. 5 enlarged, and FIG. 7 is a block diagram of a warning section according to a modification in which the first detection section and the second detection section in FIG. 2 are provided separately.

As illustrated in FIG. 1, an endoscope cleaning/disinfecting apparatus 1 includes an apparatus body 2 and a cover 3 connected to a top portion of the apparatus body 2 via, for example, a not-illustrated hinge so as to be openable/closable.

At a lower portion of the apparatus body 2, a pedal switch 2p for opening the cover 3 closed on the upper portion of the apparatus body 2 toward the upper side of the apparatus body 2 by means of an operator's operation of stepping on the pedal switch 2p is provided. Note that operation for opening/closing the cover 3 is not limited to operation of the pedal switch 2p, and, for example, the cover 3 may be opened up by the operator with his/her hand or via a switch manually operated by the operator.

In the upper portion of the apparatus body 2, a cleaning/disinfecting tank 4 that receives an endoscope 201 to be cleaned/disinfected using the endoscope cleaning/disinfecting apparatus 1 and is formed in a recess shape including a bottom face 4t and a side face 4s is provided.

Note that an opening 4k, which serves as an opening via which the endoscope 201 is received/removed in/from the cleaning/disinfecting tank 4, is openable/closable by the cover 3. The present embodiment will be indicated taking a case where one endoscope 201 is received in the cleaning/disinfecting tank 4 and the endoscope 201 is cleaned/disinfected using the endoscope cleaning/disinfecting apparatus 1, as an example; however, the present invention is not limited to this case, a configuration in which two or more endoscopes are received in the cleaning/disinfecting tank 4 and simultaneously cleaned/disinfected may be employed.

A major portion of the endoscope 201 includes, for example, an insertion portion 201a to be inserted to the inside of a subject, an operation portion 201b provided so as to be continuous with a proximal end of the insertion portion 201a, a universal cord 201c extending from the operation portion 201b and a connector portion 201d provided at an extension end of the universal cord 201c.

In the connector portion 201d, a known RF-ID 211 is incorporated. In the present invention, RF-ID is an abbreviation for radio-frequency identification and refers to one that enables sending and receiving of information via wireless communication using electromagnetic fields or radio waves.

The RF-ID 211 stores information regarding the endoscope 201 such as a model name and/or a production number of the endoscope 201. Note that if no RF-ID 211 is incorporated in the connector portion 201d, an external RF-ID tag or an external RF-ID label may be attached to the connector portion 201d. Also, a part in which the RF-ID 211 is provided is not limited to the connector portion 201d, and the RF-ID 211 may be provided in another part of the endoscope 201.

Also, inside the apparatus body 2, a warning section 100 is provided. As illustrated in FIG. 2, the warning section 100 includes: a detection section 104 including a first detection section 104a that detects reception of the endoscope 201 in the cleaning/disinfecting tank 4 and a second detection section 104b that detects removal of the endoscope 201 from the cleaning/disinfecting tank 4; a cleaning/disinfecting section 106 for performing cleaning/disinfecting of the endoscope 201 inside the cleaning/disinfecting tank 4; a cleaning/disinfecting detection section 105 that detects driving of the cleaning/disinfecting section 106; and an alerting section 107 having a function that issues a warning, and a control section 101 including a determination section 102.

Note that the control section 101 is connected at least to the cleaning/disinfecting section 106 and the alerting section 107, and is connected also to the cleaning/disinfecting detection section 105, and is further connected to the detection section 104 via a communication interface (I/F) 103.

As illustrated in FIGS. 1 and 3, the detection section 104 is provided at the bottom face 4t of the cleaning/disinfecting tank 4. Note that the detection section 104 may be provided at the side face 4s of the cleaning/disinfecting tank 4. The detection section 104 includes at least one of various types of known sensors, for example, RF-ID readers, weight sensors, optical sensors and ultrasound sensors.

In FIG. 2, an example in which the first detection section 104a and the second detection section 104b are integrally provided as the detection section 104 is illustrated; however, the present invention is not limited to this example, and as illustrated in FIG. 7, the first detection section 104a and the second detection section 104b may be separately provided.

In this case, the first detection section 104a and the second detection section 104b may be provided at different positions in the cleaning/disinfecting tank 4, or the second detection section 104b may be provided at an inner face or an outer face of the cleaning/disinfecting tank 4 and the first detection section 104a may be provided at an upper face of the apparatus body 2 as illustrated in FIG. 5.

Note that the present embodiment will be described below taking, as an example, a case where the first detection section 104a and the second detection section 104b are integrally provided as the detection section 104 as illustrated in FIG. 2 and the detection section 104 includes an RF-ID reader and is provided at the bottom face 4t of the cleaning/disinfecting tank 4 as illustrated in FIG. 1.

In other words, the detection section 104 reads the RF-ID 211 incorporated in the connector portion 201d of the endoscope 201 received in the cleaning/disinfecting tank 4 and thereby detects the reception of the endoscope 201 in the cleaning/disinfecting tank 4 and removal of the endoscope 201 from the cleaning/disinfecting tank 4.

Therefore, it is necessary that the detection section 104 be provided at a position in the bottom face 4t where the detection section 104 can read the RF-ID 211 incorporated in the connector portion 201d. Examples of a configuration that upon the endoscope 201 being put in the cleaning/disinfecting tank 4, enables the RF-ID 211 to be reliably read by the detection section 104 include the configuration illustrated in FIGS. 4 to 6.

More specifically, when the endoscope 201 is put in the cleaning/disinfecting tank 4, first, as illustrated in FIGS. 5 and 6, an endoscope guide member 250, which is illustrated in FIG. 4, is put in the cleaning/disinfecting tank 4 so that a mounting portion 250b is positioned in a reading area A for the detection section 104, which is indicated by the alternate long and two short dashes line in FIG. 6.

Next, the insertion portion 201a and the universal cord 201c are wound and mounted on a winding portion 250a of endoscope guide member 250, and the operation portion 201b and the connector portion 201d are mounted on the mounting portion 250b.

Note that the endoscope 201 may be put in the cleaning/disinfecting tank 4 together with the endoscope guide member 250 after the endoscope 201 is mounted on the endoscope guide member 250 first.

Here, as illustrated in FIG. 6, the reading area A for the detection section 104 is set to be larger in size than a placement area B for placement of the connector portion 201d in the cleaning/disinfecting tank 4, which is defined by the endoscope guide member 250. Also, a reading depth of the detection section 104 for reading the RF-ID 211 meets a depth of the cleaning/disinfecting tank 4.

Consequently, a configuration in which upon the endoscope 201 being put in the cleaning/disinfecting tank 4 using the endoscope guide member 250, enables the RF-ID 211 provided in the connector portion 201d to be reliably read by the detection section 104 is provided.

Note that the above applies to the case where the first detection section 104a and the second detection section 104b are separately provided as illustrated in FIG. 7. Positions of disposition of the first detection section 104a and the second detection section 104b in the case where the first detection section 104a and the second detection section 104b are separately provided can appropriately be determined according to, e.g., a position of placement of the endoscope.

For example, the first detection section 104a can be disposed at the position indicated by 104a in FIG. 5 and the second detection section can be disposed at a position where when the endoscope is received in the cleaning/disinfecting tank, the part of the endoscope that incorporates the RF-ID is placed. In this case, the second detection section may be disposed at either the inner face or the outer face of the cleaning/disinfecting tank.

The position of disposition of the second detection section is different depending on the range of sensing by the second detection section, and the second detection section can be disposed, for example, at the part enclosed by dotted line X in FIG. 5.

The second detection section 104b can consistently detect removal of the endoscope 201 at least while the endoscope 201 is received in the cleaning/disinfecting tank 4.

Specific examples include, e.g., the case where in a state in which the endoscope 201 is received in the cleaning/disinfecting tank 4, the second detection section 104b can consistently detect removal of the endoscope 201 while power of the endoscope cleaning/disinfecting apparatus 1 is on and the case where the second detection section 104b can consistently detect removal of the endoscope 201 while the cover 3 is opened.

For example, the cleaning/disinfecting section includes a chemical storage section that stores a chemical and a chemical supply section that supplies the chemical stored in the chemical storage section to the cleaning/disinfecting tank 4 as minimum components. The chemical referred to here may be a cleaning liquid that removes organic dirt adhering to the endoscope or may be a disinfecting liquid that detoxifies fungi adhering to the endoscope.

For example, the chemical storage section may include one or more of a detergent tank 11a, an alcohol tank 11b and a chemical tank 58, which will be described in detail below. For example, the chemical supply section may include one or more of a set of a cleaner duct 39 and a pump for detergent, a set of an alcohol duct 41 and an alcohol supply pump 42, and a set of a chemical duct 64 and a chemical pump 65.

As an example, the cleaning/disinfecting section 106 includes the part indicated by the alternate long and short dash line in FIG. 3. An example of an internal configuration of the endoscope cleaning/disinfecting apparatus 1 will be indicated below.

As illustrated in FIG. 3, the endoscope cleaning/disinfecting apparatus 1 includes a configuration that supplies tap water when one end of a water supply hose 31a is connected to a water supply hose connection port 31 and the other end of the water supply hose 31a is connected to an external faucet 5.

The water supply hose connection port 31 communicates with one end of a water supply duct 9. The other end of the water supply duct 9 is connected to a three-way solenoid valve 10, and a water supply solenoid valve 15, a check valve 16 and a water supply filter 17 are interposed in the this order from the water supply hose connection port 31 side at respective positions partway through the duct.

Note that the water supply filter 17 is a cartridge type filter so that the water supply filter 17 can be replaced periodically. The water supply filter 17 removes foreign matter and bacteria, etc., from the tap water passing through the water supply filter 17.

The three-way solenoid valve 10 is connected to one end of a flow liquid duct 18, and switches between communication of the water supply duct 9 with a water supply circulation nozzle 24 and communication of the flow liquid duct 18 with the water supply circulation nozzle 24 via an internal valve. In other words, the water supply circulation nozzle 24 communicates with either one of the water supply duct 9 and the flow liquid duct 18 by means of switching operation of the three-way solenoid valve 10. On the other end side of the flow liquid duct 18, a flow liquid pump 19 is interposed.

A circulation port 56 arranged in the cleaning/disinfecting tank 4 is connected to one end of a circulation duct 20. The other end of the circulation duct 20 bifurcates so as to communicate with the other end of the flow liquid duct 18 and one end of a channel duct 21. The other end of the channel duct 21 communicates with each of air/water feeding/forceps ports 13a to 13d. Although not illustrated, the other end of the channel duct 21 communicates also with a non-illustrated forceps raising port.

At respective positions partway through the channel duct 21, a channel pump 26, a channel block 27 and a channel solenoid valve 28 are interposed in this order from the one end side. One end of a case duct 30, the other end of which is connected to a cleaning case 6, is connected to a part of the channel duct 21 between the channel block 27 and the channel solenoid valve 28. A relief valve 36 is interposed in the case duct 30.

A detergent nozzle 22 is connected to one end of the cleaner duct 39, and the other end of the cleaner duct 39 is connected to the detergent tank 11a. At a position partway through the cleaner duct 39, a detergent pump 40, which includes a high-pressure self-suction pump, is interposed in order to lift a cleaner from the detergent tank 11a to the cleaning/disinfecting tank 4.

The alcohol tank 11b is connected to one end of the alcohol duct 41, and the alcohol duct 41 is connected to the channel block 27 so that the alcohol duct 41 communicates with the channel duct 21 in a predetermined manner.

In the alcohol duct 41, a high-pressure alcohol supply pump 42 for lifting an alcohol from the alcohol tank 11b to the cleaning/disinfecting tank 4 and a solenoid valve 43 are interposed.

Also, one end of an air duct 44 for supplying air from an air pump 45, which includes a self-suction pump that can transfer air, is connected to the channel block 27 so that the air duct 44 communicates with the channel duct 21. The other end of the air duct 44 is connected to the air pump 45, and at respective positions partway through the air duct 44, a check valve 47, and an air filter 46 to be replaced periodically are interposed.

In a drainage port 55 of the cleaning/disinfecting tank 4, an openable/closable changeover valve 57 for discharging, e.g., the cleaner to the outside or collecting a disinfecting liquid to the chemical tank 58 according to switching operation of the valve is arranged.

The changeover valve 57 is connected to one end of a drainage duct 59, the other end of which is connected to a non-illustrated drainage hose connected to an external drainage port, and a drainage pump 60 is interposed in the drainage duct 59.

The changeover valve 57 is connected also to one end of a chemical collection duct 61, and the other end of the chemical collection duct 61 is connected to the chemical tank 58.

As described above, the chemical tank 58 is connected also to one end of a chemical reception section 62 so that a chemical, for example, a disinfecting liquid is supplied from a chemical bottle 90. The other end of the chemical reception section 62 is connected in a predetermined manner to a cassette tray 80 that receives the chemical bottle 90.

A part on the one-end side of the chemical duct 64 with a suction filter 63 provided at the one end is received inside the chemical tank 58 in a predetermined manner. The other end of the chemical duct 64 is connected to a disinfecting liquid nozzle 23, and the chemical pump 65, which includes a high-pressure pump, is interposed at a position partway through the chemical duct 64 in order to lift the disinfecting liquid from the chemical tank 58 to the cleaning/disinfecting tank 4.

Note that in a portion below the bottom face 4t of the cleaning/disinfecting tank 4, as described above, for example, two ultrasound transducers 52 and a heater 53 are arranged. Also, for adjustment of a temperature of the heater 53, a temperature sensor 53a is provided at a rough center of the bottom face 4t of the cleaning/disinfecting tank 4.

The heater 53 is intended to heat the disinfecting liquid that is held inside the cleaning/disinfecting tank 4 and circulates inside the apparatus, to a predetermined temperature. Note that disinfecting liquids each have a proper temperature at which a disinfecting effect of the disinfecting liquid is most likely to be exerted. The disinfecting liquid heated to the predetermined proper temperature of the disinfecting liquid by the heater 53 can effectively disinfect the endoscope 201, and outer surfaces and the respective ducts inside the apparatus body 2.

Also, the temperature sensor 53a detects a temperature of the disinfecting liquid that is held inside the cleaning/disinfecting tank 4 and circulates inside the apparatus, and transmits a result of the detection to the control section 101. Then, based on the result of the detection from the temperature sensor 53a, the control section 101 performs control to drive/stop the heater 53 so as to maintain the disinfecting liquid at the predetermined temperature.

Inside the endoscope cleaning/disinfecting apparatus 1, a power supply 71 to which power from an external AC outlet is supplied, and the control section 101 electrically connected to the power supply 71 are provided. The control section 101 receive various signals from, e.g., a main operation panel 7 (see FIG. 5), and control driving of, e.g., the respective pumps and the respective solenoid valves described above via the communication I/F 103.

Referring back to FIG. 2, the cleaning/disinfecting detection section 105 is intended to detect driving of the cleaning/disinfecting section 106 indicated by the alternate long and short dash line in FIG. 3, and includes, for example, a water level sensor, a pressure sensor, a temperature sensor and/or another sensor.

More specifically, the cleaning/disinfecting detection section 105 detects driving of the cleaning/disinfecting section 106 by, e.g., detecting supply of a liquid to the inside of the cleaning/disinfecting tank 4, a pressure inside each of various ducts described above, a temperature of the liquid inside the cleaning/disinfecting tank 4 or driving of each of various pumps described above.

The determination section 102 is connected to the detection section 104 and the cleaning/disinfecting detection section 105, and is intended to if, after reception of the endoscope 201 in the cleaning/disinfecting tank 4 is detected by the first detection section 104a, removal of the endoscope 201 from the cleaning/disinfecting tank 4 is detected by the second detection section 104b despite no detection of driving of the cleaning/disinfecting section 106 by the cleaning/disinfecting detection section 105, determine that the uncleaned/undisinfected endoscope 201 is removed from the cleaning/disinfecting tank 4.

Note that the detection of the removal of the endoscope 201 from the cleaning/disinfecting tank 4 by the second detection section 104b is determined based on discontinuation of reading of the RF-ID 211.

In the present embodiment, the determination section 102 is provided integrally with the control section 101, but may be provided separately from the control section 101.

The control section 101 is intended to, if it is determined by the determination section 102 that the uncleaned/undisinfected endoscope 201 is removed, drive the alerting section 107 to issue a warning, and includes, for example, a CPU.

The alerting section 107 is intended to issue a warning if the uncleaned/undisinfected endoscope 201 is removed, and to warn an operator of the removal of the uncleaned/undisinfected endoscope 201 from the cleaning/disinfecting tank 4 by, for example, emitting a sound, providing display, emitting light or changing a color using, e.g., the main operation panel 7 or giving off a smell.

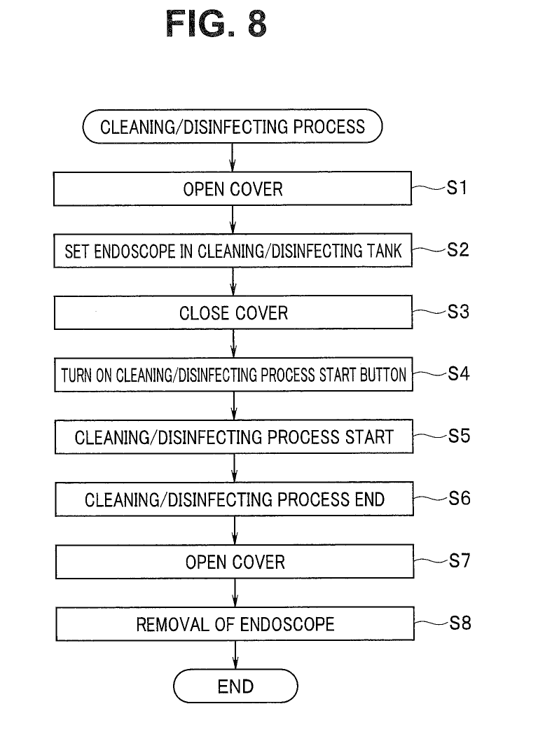

Next, a cleaning/disinfecting process for the endoscope 201 using the endoscope cleaning/disinfecting apparatus 1 in FIG. 1 will briefly be described with reference to FIG. 8. FIG. 8 is a flowchart schematically indicating a cleaning/disinfecting process for an endoscope using the endoscope cleaning/disinfecting apparatus in FIG. 1.

As illustrated in FIG. 8, first, in step S1, an operator opens the cover 3 by stepping on the pedal switch 2p. However, S1 is not an essential step because, depending on the facility, the cover 3 of the cleaning/disinfecting tank may be left open until next endoscope cleaning/disinfecting after previous endoscope cleaning/disinfecting.

Next, in step S2, the used endoscope 201 is put and thereby received in the cleaning/disinfecting tank 4 as described above with reference to FIGS. 4 to 6. After the reception, the RF-ID 211 is consistently read by the detection section 104.

The endoscope 201 is set by connecting the respective air/water feeding/forceps ports 13a to 13d provided in the cleaning/disinfecting tank 4 and a tube extending from a non-illustrated water leakage detection port to non-illustrated fittings provided at the operation portion 201b and the connector portion 201d.

Subsequently, in step S3, the cover 3 is closed. Note that at this point of time, the cover 3 is not locked and the cover 3 can be opened again. In other words, the operator can remove the uncleaned/undisinfected endoscope 201 from the cleaning/disinfecting tank 4.

This is because after the cover 3 is closed, the cover 3 may be opened again to adjust a position of placement of the endoscope 201 in the cleaning/disinfecting tank 4 again or to put a second endoscope in the cleaning/disinfecting tank.

Next, in step S4, a cleaning/disinfecting process start button 7s provided on the main operation panel 7 is turned on.

Note that after the cleaning/disinfecting process start button 7s is turned on, the cover 3 is locked and is prevented from being unlocked until end of the cleaning/disinfecting process in step S6.

In other words, after start of the cleaning/disinfecting process in step S5, until the end in step S6, the operator cannot remove the endoscope 201 from the cleaning/disinfecting tank 4 unless the operator forcibly turns off the power of the endoscope cleaning/disinfecting apparatus 1. Therefore, after the start of the cleaning/disinfecting process, the reading of the RF-ID 211 by the detection section 104 may be stopped in consideration of, e.g., energy saving.

As a result, in step S5, a known cleaning/disinfecting process for cleaning/disinfecting the endoscope 201 in the cleaning/disinfecting tank 4 starts, and after a lapse of a fixed time period, in step S6, the cleaning/disinfecting process ends. Note that after the end of the cleaning/disinfecting process, the cover 3 is unlocked.

Subsequently, in step S7, the operator opens the cover 3 by stepping on the pedal switch 2p and removes the cleaned/disinfected endoscope 201 from the cleaning/disinfecting tank 4.

Subsequently, the control section 101 clears the RF-ID information read by the detection section 104. Note that the clearance of the RF-ID information may be performed after the end of the cleaning/disinfecting process.

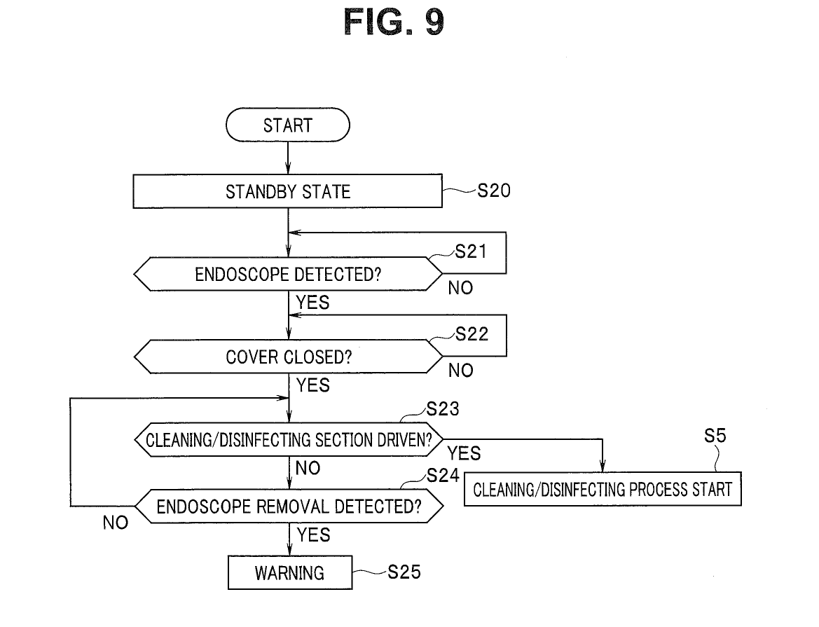

Next, control operation performed by the control section 101 in FIG. 2 to control the warning section 100 will be described with reference to FIG. 9. FIG. 9 is a flowchart indicating control operation performed by the control section in FIG. 2 to control the warning section.

As illustrated in FIG. 9, first, in a standby state in step S20 in which the detection section 104 can detect the endoscope 201, in step S21, whether or not the endoscope 201 is received in the cleaning/disinfecting tank 4 is determined, that is, whether or not the first detection section 104a detects the endoscope 201 is determined using the first detection section 104a. More specifically, whether or not the endoscope 201 is received is determined according to whether or not the RF-ID 211 is read by the first detection section 104a.

If the endoscope 201 is detected, in step S22, whether or not the cover 3 is closed is determined.

If the cover 3 is closed, in step S23, whether or not the cleaning/disinfecting section 106 is driven is determined using the cleaning/disinfecting detection section 105.

If the cleaning/disinfecting section 106 is driven, the operation proceeds to step S5, and the above-described cleaning/disinfecting process is started. If the cleaning/disinfecting section 106 is not driven, the operation proceeds to step S24, and whether or not the endoscope 201 is removed from the cleaning/disinfecting tank 4 before start of a cleaning/disinfecting process is determined using the second detection section 104b. More specifically, whether or not the endoscope 201 is removed from the cleaning/disinfecting tank 4 is detected according to whether or not the reading of the RF-ID 211 by the second detection section 104b is discontinued.

If removal of the endoscope 201 is not detected, more specifically, if the reading of the RF-ID 211 by the second detection section 104b is continued, it is determined that that the endoscope 201 is continuously received in the cleaning/disinfecting tank 4 and the operation returns to step S23.

On the other hand, if removal of the endoscope 201 is detected, more specifically, if the reading of the RF-ID 211 by the second detection section 104b is discontinued, it is determined by the determination section 102 that the endoscope 201 is removed from the cleaning/disinfecting tank 4.

Note that the determination of the removal of the endoscope 201 from the cleaning/disinfecting tank 4 by the determination section 102 is made immediately after the detection of removal of the endoscope 201 by the second detection section 104b, that is, immediately after the discontinuation of the reading of the RF-ID 211.

Lastly, in step S25, the alerting section 107 is driven to issue a warning to warn the operator of the removal of the uncleaned/undisinfected endoscope 201 from the cleaning/disinfecting tank 4.

As described above, the present embodiment indicates that the second detection section 104b can consistently detect removal of an endoscope 201 at least while the endoscope 201 is received in the cleaning/disinfecting tank 4.

More specifically, the present embodiment indicates that the second detection section 104b consistently reads the RF-ID 211 of the endoscope 201 at least while the endoscope 201 is received in the cleaning/disinfecting tank 4.

Also, if after reception of the endoscope 201 in the cleaning/disinfecting tank 4 is detected by the first detection section 104a, removal of the endoscope 201 from the cleaning/disinfecting tank 4 is detected by the second detection section 104b despite no detection of driving of the cleaning/disinfecting section 106 by the cleaning/disinfecting detection section 105, the determination section 102 determines that the uncleaned/undisinfected endoscope 201 is removed from the cleaning/disinfecting tank 4.

More specifically, if the reading of the RF-ID 211 by the second detection section 104b is discontinued, the determination section 102 determines that the uncleaned/undisinfected endoscope 201 is removed from the cleaning/disinfecting tank 4.

Furthermore, the present embodiment indicates that if it is determined by the determination section 102 that the uncleaned/undisinfected endoscope 201 is removed, the control section 101 drives the alerting section 107 to issue a warning.

Consequently, if the uncleaned/undisinfected endoscope 201 received in the cleaning/disinfecting tank 4 is removed from the cleaning/disinfecting tank 4 before start of a cleaning/disinfecting process, a warning is issued without fail, enabling an operator to easily recognize that the removed endoscope 201 is an uncleaned/undisinfected one.

According to the above, the endoscope cleaning/disinfecting apparatus 1 including a configuration that can suppress the uncleaned/undisinfected endoscope 201 being taken out from the cleaning/disinfecting tank 4 by an operator can be provided.

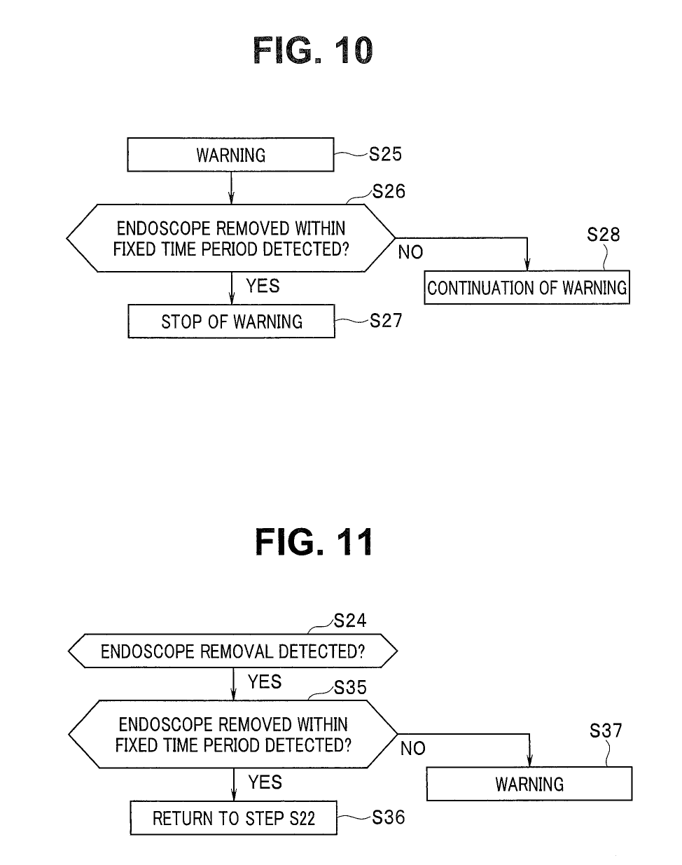

Note that a modification will be indicated below with reference to FIG. 10. FIG. 10 is a flowchart indicating a modification of control operation performed by the control section in FIG. 2 to control the warning section when an endoscope is detected again within a fixed time period after the warning step in FIG. 9.

The present embodiment described above indicates that a warning is issued from the alerting section 107 immediately after detection of removal of the endoscope 201 by the second detection section 104b, that is, immediately after discontinuation of the reading of the RF-ID 211.

However, after the cover 3 is closed in step S3 in FIG. 8 and before the cleaning/disinfecting process start button 7s is turned on in step S4, as described above, the cover 3 may be opened again to adjust a position of placement of the endoscope 201 in the cleaning/disinfecting tank 4 again.

This case has a problem that if the endoscope 201 is removed from the cleaning/disinfecting tank 4 to reset the endoscope 201, a warning is issued and even though the endoscope 201 is subsequently reset in the cleaning/disinfecting tank 4, the warning is continuously issued, which is inconvenient to the operator.

Therefore, as illustrated in FIG. 10, it is possible that upon removal of the endoscope 201 from the cleaning/disinfecting tank 4 to reset the endoscope 201 before the cleaning/disinfecting process start button 7s is turned on in step S4 in FIG. 8, in step S25, a warning is issued from the alerting section 107; and subsequently, in step S26, if the endoscope 201 that is the removed endoscope is detected again by the first detection section 104a within a fixed time period, the control section 101 proceeds to step S27 and performs control to stop the warning; and if the removed endoscope 201 is not detected within the fixed time period, in step S28, the control section 101 performs control to continue the warning.

Consequently, the warning is stopped when the operator resets the removed endoscope 201 in the cleaning/disinfecting tank 4, facilitating the work of resetting the endoscope 201. Also, the warning is continued unless the removed endoscope 201 is reset, enabling prevention of use of the uncleaned/undisinfected endoscope.

Another modification will be indicated below with reference to FIG. 11. FIG. 11 is a flowchart indicating a modification of the control operation performed by the control section in FIG. 2 to control the warning section when an endoscope is detected again within a fixed time period after the endoscope removal detection step in FIG. 9.

The present embodiment described above indicates that determination that the endoscope 201 is removed from the cleaning/disinfecting tank 4 by the determination section 102 is made immediately after detection of removal of the endoscope 201 by the second detection section 104b, that is, immediately after discontinuation of reading of the RF-ID 211; and a warning is issued from the alerting section 107.

However, as described above, after the cover 3 is closed in in step S3 in FIG. 8 and before the cleaning/disinfecting process start button 7s is turned on in step S4, the cover 3 may be opened again to adjust a position of placement of the endoscope 201 in the cleaning/disinfecting tank 4 again. In this case, even though the endoscope 201 is removed from the cleaning/disinfecting tank 4 just to reset the endoscope 201, a warning is issued.

Therefore, the determination section 102 may be configured to, only if reception of the endoscope 201 that is the removed endoscope is not detected by the first detection section 104a after a lapse of a fixed time period after detection of removal of the endoscope 201 by the second detection section 104b, determine that the uncleaned/undisinfected endoscope 201 is removed, and the control section 101 may provide a warning using the alerting section 107 only in this case.

More specifically, as illustrated in FIG. 11, the configuration may be made so that a warning is not issued immediately after the detection of the removal of the endoscope in step S24 and the operation process to step S35, and if the removed endoscope 201 is detected by the first detection section 104a within a fixed time period, it is determined that the endoscope 201 is reset and the operation proceeds to step S36 to return to step S22 in FIG. 9 without issuance of a warning, and only if the removed endoscope 201 is not detected by the first detection section 104a within the fixed time period, it is determined that the uncleaned/undisinfected endoscope 201 is taken out of the cleaning/disinfecting tank 4 and the operation process to step S37 to issue a warning.

Consequently, even if the operator removes the endoscope 201 to reset the endoscope 201 before a cleaning/disinfecting process, no warning is issued, and no warning is also issued if the operator returns the endoscope 201 to the cleaning/disinfecting tank 4 within the fixed time period, facilitating the resetting work.

Also, if the operator does not return the removed endoscope 201 within the fixed time period, a warning is provided, enabling prevention of use of the uncleaned/undisinfected endoscope.

### Second Embodiment

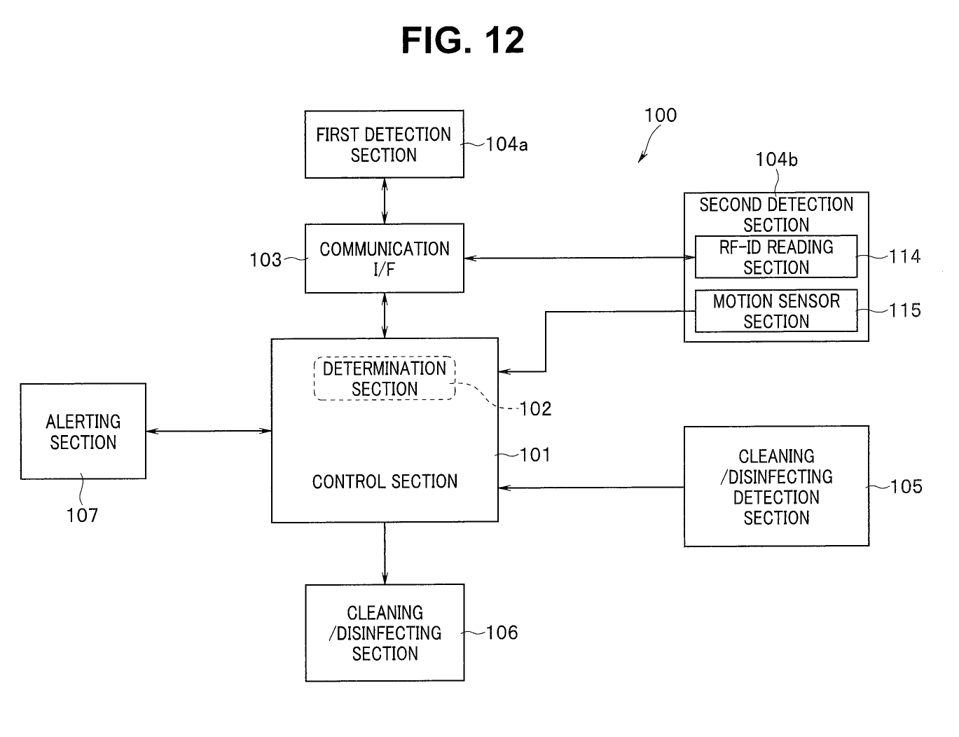

FIG. 12 is a block diagram schematically illustrating a warning section inside an apparatus body of an endoscope cleaning/disinfecting apparatus according to the present embodiment.

The endoscope cleaning/disinfecting apparatus according to the second embodiment is different from the endoscope cleaning/disinfecting apparatus according to the above-described first embodiment in that a first detection section and a second detection section are separately provided; and the second detection section includes an RF-ID reading section and a motion sensor section.

Therefore, description will be provided only on these differences, components that are the same as those of the first embodiment will be provided with reference numerals that are the same as those of the first embodiment and description of such components will be omitted.

As illustrated in FIG. 12, in a warning section 100 of an endoscope cleaning/disinfecting apparatus 1 according to the present embodiment, as mentioned above with reference to FIG. 7, a first detection section 104a and a second detection section 104b are separately provided, and the second detection section 104b includes an RF-ID reading section 114 that reads an RF-ID 211 of an endoscope 201, and a motion sensor section 115 that detects motion of an operator.

Note that a motion sensor in the present invention refers to one that has a predetermined sensing area, reacts to an object entering the sensing area and continuously reacts to the object if the object stays within the sensing area after the entry.

Note that although the above-described first embodiment indicates that the second detection section 104b can consistently detect removal of the endoscope 201 at least while the endoscope 201 is received in the cleaning/disinfecting tank 4, in the present embodiment, the RF-ID reading section 114 in the second detection section 104b continues reading the RF-ID 211 of the endoscope 201 received in a cleaning/disinfecting tank 4 only while the presence of the operator within a predetermined area from the cleaning/disinfecting tank 4 is continuously detected by the motion sensor section 115.

This is because in order to remove the uncleaned/undisinfected endoscope 201 from the cleaning/disinfecting tank 4, an operator inevitably needs to approach the endoscope cleaning/disinfecting apparatus 1 and thus the RF-ID reading section 114 does not need to continuously and consistently read the RF-ID 211 while the endoscope 201 is received in the cleaning/disinfecting tank 4, and reading of the RF-ID 211 only when presence of the operator is detected by the motion sensor section 115 enables detection of removal of the uncleaned/undisinfected endoscope 201.

Note that the RF-ID reading section 114 may be configured to, even if presence of the operator is detected by the motion sensor section 115, stop reading of the RF-ID 211 of the endoscope 201 received in the cleaning/disinfecting tank 4 when a cover 3 is closed. In other words, the RF-ID reading section 114 may be configured to read the RF-ID 211 only when the cover 3 is opened and presence of the operator is detected by the motion sensor section 115. This is because when the cover 3 is closed, there is no possibility of the endoscope 201 being removed from the cleaning/disinfecting tank 4.

Note that warning control performed by the control section 101 in relation to removal of the uncleaned/undisinfected endoscope 201 from the cleaning/disinfecting tank 4 is the same as the warning control described above with reference to FIGS. 9 to 11.

With such configuration, use of the motion sensor section 115 enables reduction in power consumed by the endoscope cleaning/disinfecting apparatus 1 compared to the configuration in which the second detection section 104b continues reading the RF-ID 211 while the endoscope 201 is received in the cleaning/disinfecting tank 4 as in the above-described first embodiment. Note that the rest of the effects is the same as that of the above-described first embodiment.

Here, after the end of the cleaning/disinfecting process in step S6 in FIG. 8, if the cover 3 is left unopened for a long period of time, the operator cannot recognize whether or not the endoscope 201 received in the cleaning/disinfecting tank 4 is a cleaned/disinfected one and may clean/disinfect the cleaned/disinfected endoscope 201 again.

Therefore, the control section 101 may be configured so that if, after the end of the cleaning/disinfecting process in step S6, the cleaning/disinfecting process start button is turned on again in a state in which the RF-ID 211 is continuously read by the detection section 104 or the second detection section 104b, inform the operator of the endoscope 201 received in the cleaning/disinfecting tank 4 being a cleaned/disinfected one by providing a warning using the alerting section 107 without staring the cleaning/disinfecting process, because the detection section 104 or the second detection section 104b continue reading the RF-ID 211 and it is thus recognized that the endoscope 201 is a cleaned/disinfected one.

Note that the above is premised on the assumption that even after the end of the cleaning/disinfecting process, the detection section 104 or the second detection section 104b continue reading the RF-ID 211 of the cleaned/disinfected endoscope 201.

What is claimed is:

1. An endoscope cleaning/disinfecting apparatus comprising:
a cleaning/disinfecting tank that is a container receiving an endoscope;
a first detection section that detects reception of the endoscope by the cleaning/disinfecting tank;
a second detection section that detects removal of the endoscope from the cleaning/disinfecting tank;
a cleaning/disinfecting section for cleaning/disinfecting the endoscope inside the cleaning/disinfecting tank;
a cleaning/disinfecting detection section that detects driving of the cleaning/disinfecting section;
an alerting section that issues a warning;
a determination section that is connected to the first detection section, the second detection section and the cleaning/disinfecting detection section, and if, after reception of the endoscope in the cleaning/disinfecting tank is detected by the first detection section, removal of the endoscope from the cleaning/disinfecting tank is detected by the second detection section despite no detection of driving of the cleaning/disinfecting section by the cleaning/disinfecting detection section, determines that the endoscope which is uncleaned/undisinfected is removed from the cleaning/disinfecting tank; and
a control section that is connected to at least the determination section and the alerting section, and if it is determined by the determination section that the uncleaned/undisinfected endoscope is removed, drives the alerting section to issue a warning.

2. The endoscope cleaning/disinfecting apparatus according to claim 1, wherein the second detection section is provided at an inner face of the cleaning/disinfecting tank or a back face of the cleaning/disinfecting tank, and can consistently detect removal of the endoscope at least while the endoscope is received in the cleaning/disinfecting tank.

3. The endoscope cleaning/disinfecting apparatus according to claim 2, further comprising a cover that is openable/closable relative to the cleaning/disinfecting tank,
wherein the second detection section consistently detects removal of the endoscope from the cleaning/disinfecting tank while the cover is opened.

4. The endoscope cleaning/disinfecting apparatus according to claim 1, wherein the control section drives the alerting section within a predetermined time period from detection of the removal of the endoscope by the second detection section.

5. The endoscope cleaning/disinfecting apparatus according to claim 1, wherein if reception of the endoscope is not detected by the first detection section after a lapse of a fixed time period from detection of removal the endoscope by the second detection section, the determination section determines that the uncleaned/undisinfected endoscope is removed.

6. The endoscope cleaning/disinfecting apparatus according to claim 1, wherein:
the second detection section includes a motion sensor section and an RF-ID reading section that reads an RF-ID of the endoscope received in the cleaning/disinfecting tank; and
the RF-ID reading section continuously reads the RF-ID of the endoscope received in the cleaning/disinfecting tank while the motion sensor section is reacting.

7. The endoscope cleaning/disinfecting apparatus according to claim 6, further comprising a cover that is openable/closable relative to the cleaning/disinfecting tank,
wherein upon closure of the cover, the RF-ID reading section stops the reading of the RF-ID of the endoscope received in the cleaning/disinfecting tank.
